(19) United States
(12) Patent Application Publication
(10) Pub. No.: US 2016/0031909 A1
(43) Pub. Date: Feb. 4, 2016

(54) PORPHYRAZINE OPTICAL AND DUAL OPTICAL/ MR CONTRAST AND THERAPEUTIC AGENTS

(71) Applicant: HOFFMAN/BARRETT, LLC, Evanston, IL (US)

(72) Inventors: Evan R. Trivedi, Floyds Knobs, IN (US); Emily A. Waters, Mount Prospect, IL (US); Anthony G.M. Barrett, London (GB); Brian M. Hoffman, Evanston, IL (US); Thomas J. Meade, Wilmette, IL (US)

(21) Appl. No.: 14/774,261

(22) PCT Filed: Mar. 6, 2014
(86) PCT No.: PCT/US14/20977

§ 371 Date: Sep. 10, 2015

Related U.S. Application Data
(60) Provisional application No. 61/777,280, filed on Mar. 12, 2013.

(51) Int. Cl. C07F 5/00; A61K 49/00; A61N 5/10; A61K 45/06; A61N 5/06; A61K 49/10; A61K 41/00

(52) U.S. Cl. CPC C07F 5/003; A61K 49/108; A61K 49/0036; A61K 41/0071; A61K 45/06; A61N 5/062; A61N 5/10

(57) ABSTRACT

Porphyrazines capable of localizing in a tumor of a mammal are disclosed. The porphyrazines are used in methods of imaging a tumor and in methods of treating tumors, either alone or in combination with a chemotherapeutic agent and/or radiation.

### STATEMENT OF GOVERNMENTAL INTEREST

This application was supported by grant number RO1EB005866 awarded by the National Institutes of Health. The government has certain rights in the invention.

### FIELD OF THE INVENTION

The present invention relates to porphyrazine Gd(III) conjugates, and the use of the porphyrazine Gd(III) conjugates in the imaging and treatment of tumors. The porphyrazine Gd(III) (pz-Gd) conjugates are capable of localizing in a tumor, and permit detection of the tumor by visualization techniques, such as near infrared (NIR) and magnetic resonance (MR) imaging. The pz-Gd conjugates also reduce the size of, or eliminate, tumors when administered with light activation.

### BACKGROUND OF THE INVENTION

Survival rates of breast cancer patients could be improved if tumors are detected in their early stages or, following treatment such as chemotherapy, surgery, or radiation, residual cancer cells easily could be detected at the cellular level. The primary technique for early screening for breast cancer, i.e., X-ray mammography, is effective, but has disadvantages. The differentiation between normal tissue and cancerous tissue based on their relative density is small, causing false-positives and subjecting patients to additional testing that may be unnecessary, invasive, and often painful. Furthermore, exposure to ionizing radiation inherent to x-ray procedures limits the frequency that high-risk patients can be screened. The limited sensitivity of this technique allows small, early-stage tumors to be missed, as well as the failure to recognize residual cancer cell clusters after treatment and to clearly define the margins of the cancer thereby permitting the disease to progress and reemerge and between screenings Implementation of new, sensitive, and safe detection methods would improve the diagnosis of breast cancer and prognosis of breast cancer patients by reducing levels of morbidity and mortality.

Fluorescence imaging using near-infrared (NIR) contrast agents is an emerging, highly sensitive method for tumor detection that takes advantage of the relative transparency of mammalian tissue to NIR light (about 700-1000 nm). A contrast agent that absorbs and emits light in the NIR range and accumulates specifically in tumor tissue could be optically imaged through soft tissue, thereby providing an ideal, non-invasive detection method for superficial tumors in soft tissue, such as those of the breast. Luminescence imaging of soft tissue with light at near-infrared (NIR) wavelengths within the window of relative tissue transparency therefore represents an important emerging method of tumor detection, but, as with other imaging modalities, effective contrast agents are needed.

Magnetic resonance imaging (MRI) is an important diagnostic tool for imaging soft tissues that offers high resolution and deep tissue penetration (1). Most important is the absence of harmful ionizing radiation inherent to more common X-ray procedures, allowing more frequent or long-term MRI scans (2). The replacement of X-ray mammography with MRI for breast cancer screening has therefore been examined (3). Without the use of a contrast agent, MRI relies heavily on varying tissue density, and therefore water content, to differentiate between structures. Tumors have a wide range of tissue morphology that cannot always be differentiated from surrounding tissue by MRI, and as such, MRI is not effective in clearly defining tumor margins or tissues that are magnetically similar but histologically distinct. Therefore, one potential avenue to improve MRI for cancer diagnosis is to implement the use of a tumor-specific molecular contrast agent, such as a paramagnetic Gd(III) complex, that would highlight tumor tissue regardless of its apparent similarity to surrounding healthy tissue (4).

Hydrophilic Gd(III) contrast agents primarily are used clinically for brain angiography because they are restricted to the vascular space, and therefore can identify damaged blood vessels (5). This concept can be applied to tumor imaging, because most tumors are more perfuse than surrounding tissue. However, a tumor-specific contrast agent capable of crossing cell membranes would be a desirable advance in the art. Strategies for achieving such a contrast agent include covalently attaching tumor specific biomolecules, such as steroids or peptides (6), or altering amphiphilicity by adding a hydrophobic moiety (6a, 6b, 7). One such hydrophobic class of molecules are tetrapyrroles, which have been extensively studied for molecular imaging applications (8). Specific porphyrinoid complexes with Gd(III) for MRI applications have been reported (9), representing the viability of this approach.

Porphyrazines (pzs) are a sub-class of tetrapyrrole macrocycles that have been examined for a number of tumor biology applications including photodynamic therapy (PDT) and near infrared (NIR) optical imaging (10). Porphyrazines exhibit a combination of photophysical, chemical, and biological properties that make them attractive optical tumor-imaging agents. The hydrophobic nature and lack of aggregation in solution of one such macrocycle, i.e., pz 247, imparts preferential tumor uptake due to association and co-transport with low-density lipoprotein (LDL) via LDL-receptor (LDLr) mediated endocytosis (11).

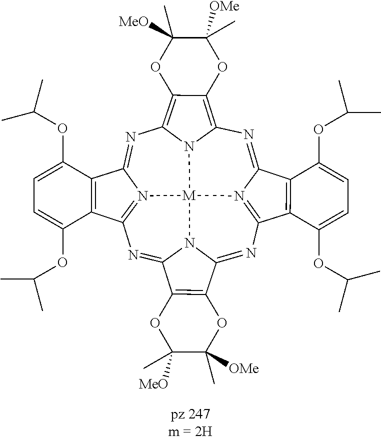

This tumor specific uptake mechanism theoretically lies in the hyperproliferative tumor tissue's greater need for lipids and cholesterol to build new cell membranes. The hydrophobicity of a pz leading to tumor cell uptake resulted in the development of first generation pz-Gd(III) conjugates as potential MRI contrast agents (12). One of these conjugates was taken up by tumor cells in vitro, but poor synthetic yields precluded further development and testing in vivo.

The present invention is directed to the design, synthesis, and characterization of novel second generation pz-Gd(III) conjugates. The present compounds are taken up by cells in vitro warranting extensive in vivo MRI studies in athymic nude mouse tumor models.

### SUMMARY OF THE INVENTION

The present invention is directed to pz-Gd(III) conjugates and use of the pz-Gd(III) conjugates in imaging and detecting tumors in a mammal, and in the size reduction and elimination of such tumors.

Near infrared (NIR) optical imaging and MR imaging are precise, non-invasive techniques for breast cancer diagnosis and the diagnosis of other cancers in soft tissue, including skin and testicular cancers, and cancers detected using endoscopic devices. The porphyrazine compounds of the present invention greatly improve early stage and post remedial intervention cancer detection. Therefore, one aspect of the present invention is to provide pz-Gd(III) conjugates capable of localizing in a tumor. The present pz-Gd(III) conjugates demonstrate tumor-cell uptake in vitro, and also exhibit tumor-specific accumulation and retention in subcutaneous tumors in vivo.

The present invention includes embodiments in which (a) the pz-Gd(III) conjugate acts as an NIR or MR imaging agent to detect tumors, and (b) the pz-Gd(III) conjugate exhibits a toxicity with respect to tumor cells and therefore can reduce the size of, or eliminate, the tumor when administered with light activation.

Porphyrazines (pzs), or tetraazaporphyrins, are being studied for their potential use in detection and treatment of cancers. An amphiphilic Cu(II)-Pz-Gd(III) conjugate has been prepared via cycloaddition “click” chemistry between an azide-functionalized pz and alkyne functionalized DOTA-Gd(III) analog for use as an MRI contrast agent. The Cu-Pz-Gd(III) conjugate is synthesized in good yield and exhibits solution-phase ionic relaxivity (r₁=11.5 mM⁻¹s⁻¹) that is about four times greater than that of a clinically-used monomeric Gd(III) conjugate contrast agent, DOTA-Gd(III). Breast tumor cells (MDA-MB-231) take up the Cu-Pz-Gd(III) conjugate in vitro where significant contrast enhancement (9.336±0.335 CNR) is observed in phantom cell pellet MR images. This novel contrast agent was administered in vivo to an orthotopic breast tumor model in athymic nude mice and MR images were collected. The average T₁ of tumor regions in mice treated with 50 mg/kg of the Cu-Pz-Gd(III) conjugate decreased relative to saline-treated controls. Furthermore, the decrease in T₁ was persistent relative to mice treated with the monomeric Gd(III) contrast agent. An ex vivo biodistribution study confirmed that Cu-Pz-Gd(III) conjugate accumulates in tumors and is rapidly cleared, primarily through the kidneys. Differential accumulation and T₁ enhancement by the Cu-Pz-Gd(III) conjugate in the tumor core relative to the periphery offer evidence that this compound has application in the imaging of necrotic tissue.

In various aspects, the present invention provides pz-Gd(III) conjugates useful as an MRI contrast agent for (a) tumor diagnosis, (b) tumor treatment monitoring, (c) imaging tumor-necrosis, and (d) imaging of necrotic tissue resulting from myocardial infarction.

Another aspect of the present invention is to provide a method of detecting a tumor or necrotic tissue in a mammal by administering a sufficient amount of a pz-Gd(III) conjugate of the present invention for visualization to an individual, then visualizing the pz-Gd(III) conjugate in the mammal. The visualizing can be NIR imaging or MR imaging. In this aspect of the invention, the two imaging methods can be used after the administration of a single pz-Gd(III) conjugate of the present invention.

Still another aspect of the present invention is to provide a method of treating an individual having a tumor, wherein a pz-Gd(III) conjugate is administered to the individual in a sufficient amount to localize in the tumor and kill tumor cells with light activation.

In other aspects, embodiments of the present invention further provide kits and methods of use of the pz-Gd (III) conjugates in imaging for research, diagnostic, and clinical applications.

These and other novel aspects of the present invention will become apparent from the following detailed description of the preferred embodiments.

### BRIEF DESCRIPTION OF THE FIGURES

FIG. 1 contains bar graphs of fmoles Gd(III)/cell vs incubation concentration [Gd(III)] μm showing the dose dependent uptake of M-Pz-Gd(III) complexes in MDA-MB-231 cells, compared to monomeric DOTA-Gd(III) as a positive control;

FIG. 2—(A) T₁-weighted MR image with corresponding image-intensity color map of MDA-MB-231 cells incubated with Cu-Pz-Gd(III), Zn-Pz-Gd(III), DOTA-Gd(III), and media alone for 24 hours (scale bar=0.5 mm) 9.4 T (400 MHz) and 25° C. (TR/TE=500/10 ms); (B) Calculated values corresponding to the MR image in (A);

FIG. 3—(A) Transverse slices through implanted tumors in mice treated with 50 mg/kg Cu-Pz-Gd(III) (upper) and saline (lower). T₁ maps of tumor region overlaid onto anatomical images. (B) Ex vivo Gd(III) content in tumor tissue as measured by ICP-MS over time in mice treated with 400 nmol Cu-Pz-Gd(III).;

FIG. 4 contains selected images from the 3.5 hour time course. The left column shows raw T₂ weighted images of the center slice through the tumor. The next four columns show the T₁ map of the tumor overlaid on the corresponding T₂ weighted image at baseline and at 10, 90, and 180 minutes after administration of imaging agent. The T₁ becomes progressively shorter (indicating Gd(III) accumulation) in the Cu-Pz-Gd(III) treated animal, whereas it dips transiently but returns quickly to baseline in the DOTA-Gd(III) treated animal, and remains constant in the saline treated animal; and

FIG. 5—(A,B) T₁ over time for tumor regions of interest after injection of 450 nmol Cu [symbol] Pz-Gd(III), showing the steady accumulation of Cu-Pz-Gd(III) in tumor tissue with greater accumulation in the necrotic core. DOTA-Gd(III) enters the tumor, but is cleared relatively rapidly. Very little change is observed in the saline treated animals, indicating repeatability of the measurements. (C) T₂ weighted image of untreated tumor showing the two distinct ROI that were chosen. (D) Histological image of tissue from the tumor core. (E) Histological image of tissue from the tumor periphery. Fenestrations are present at the tumor's core indicating potential necrosis.

### DETAILED DESCRIPTION OF THE PREFERRED EMBODIMENTS

The present invention is directed to pz-Gd(III) conjugates that exhibit a combination of optical, chemical, and biological properties making them uniquely attractive as tumor-imaging agents and as a platform for MRI imaging agents. Test results show that the present pz-Gd(III) conjugates exhibit intense optical absorbance and fluorescence in the NIR window with optimum tissue penetration and are selectively taken up by tumor cells and by tumors in vivo.

In some embodiments, the present pz-Gd(III) conjugates also are capable of reducing the size of and/or eliminating tumors with light activation. The present invention therefore relates to pz-Gd(III) conjugates and their use as NIR/MR imaging agents. Embodiments of the present invention include the use of the present pz-Gd(III) conjugates as imaging therapeutic agents.

Heteroatom-functionalized porphyrazines (pzs) and their derivatives are porphyrinoid macrocycles that have been investigated as optical contrast agents for tumor detection and as a platform for cancer treatment using photodynamic therapy (PDT). These pzs have intense NIR absorption/emission and are synthetically flexible, making it possible to design a pz having desirable NIR optical characteristics, while independently adjusting amphiphilicity and cell recognition, properties that dictate tumor-specific retention of the porphyrazines.

Porphyrazines (pzs) are aromatic macrocycles different from porphyrins in that the meso carbons are replaced with nitrogen. The meso-nitrogen atoms confer intense NIR absorption and emission, thereby making pzs excellent photosensitizers for the detection of superficial cancers via optical imaging. Compounds of the present invention therefore are derivatives of the following macrocyclic core, abbreviated herein as “pz-2H” with substitution of 2H by metal ions and functionalization of the macrocycle periphery:

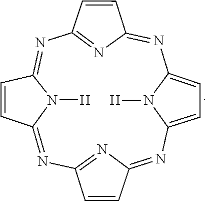 [SMILES: C1=Cc2nc1nc1ccc(nc3nc(nc4ccc(n2)[nH]4)C=C3)[nH]1]

Pz-Gd(III) conjugates of the present invention have a general structure:

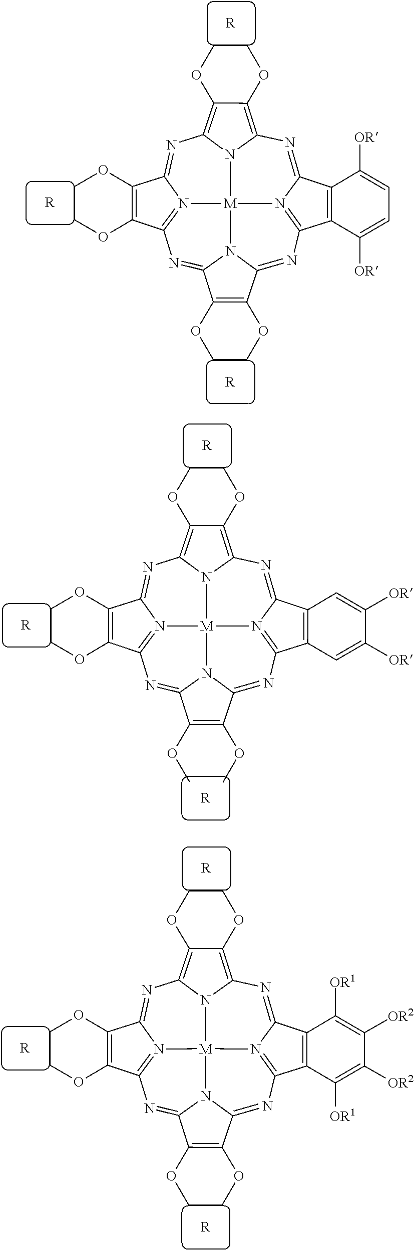

wherein M is 2H, Cu, Mg, or Zn;

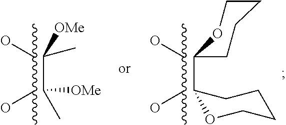

independently, is

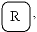

R′ is

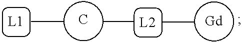

R¹ and R², each independently, are R′, OC₁₋₈alkyl, C₃₋₈cycloalkyl, O(CH₂CH₂O)ₙOH, or O(CH₂CH₂O)ₙO(C₁₋₄alkyl);

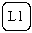

is (CH₂)ₙ, (CH₂CH₂O)ₙ, OOC(CHOH)ₙCOO, COO(CH₂)ₙOOC, or CON(CH₂)ₙNOC, wherein n is 1 through 10;

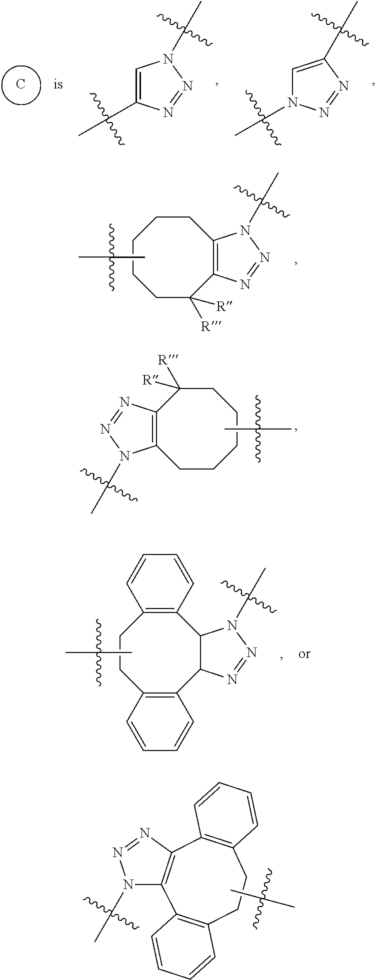

wherein R″ and R′″, independently, are H or F; and

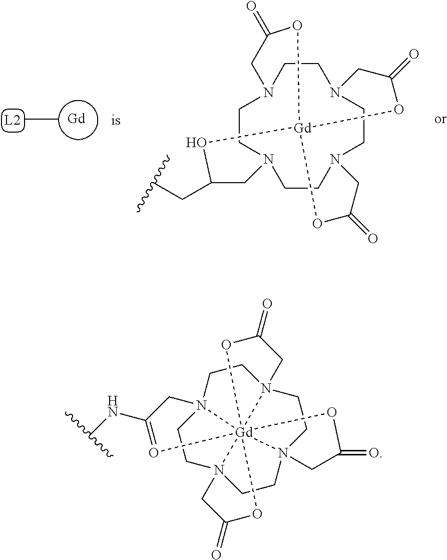

### Synthesis

The pz platform is diol pz 285, which was prepared and activated for nucleophilic substitution by arenesulfonylation to produce bis-tosylate pz 286 as previously described (13). Subsequent nucleophilic substitution of pz 286 with excess sodium azide gave diazide pz 288, which was suitable for conjugation via cycloaddition reactions with the alkyne-functionalized Gd595. (Scheme 1). The use of catalytic copper(II) sulfate in this conjugation reaction partially metallates the pz core. To address this issue, pz 288 either was metallated with ZnCl₂ prior to reaction with the alkyne or a stoichiometric amount of the Cu(SO₄)₂ catalyst was used to ensure that the pz core was completely cuprated. Standard cycloaddition reaction conditions were subsequently used to conjugate metallated pz diazides with Gd595 to produce Zn-Pz-Gd(III) and Cu-Pz-Gd(III) conjugates, which were isolated by preparative HPLC in 52% and 57% isolated yields, respectively. Trace amounts of Zn-Pz-Gd(III) were transmetallated to Cu-Pz-Gd(III), but this small amount was removed during purification. The resulting Zn-Pz-Gd(III) and Cu-Pz-Gd(III) conjugates are freely water soluble, allowing intravenous administration without the addition of a co-solvent or other formulation components.

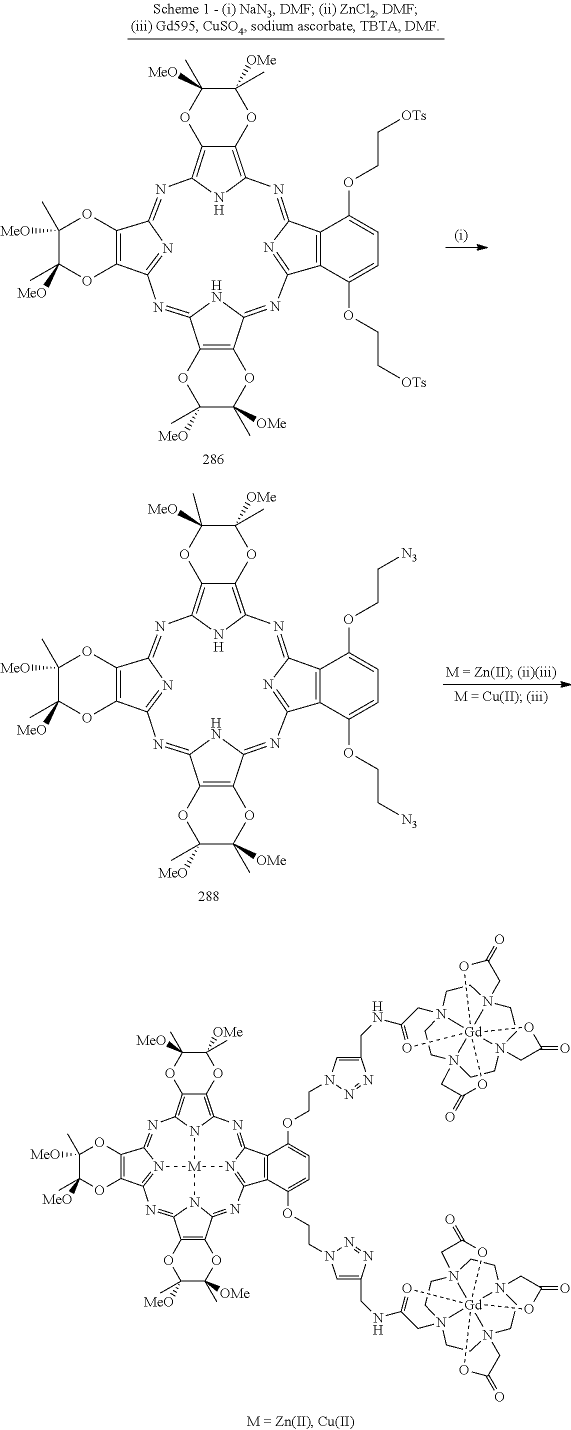

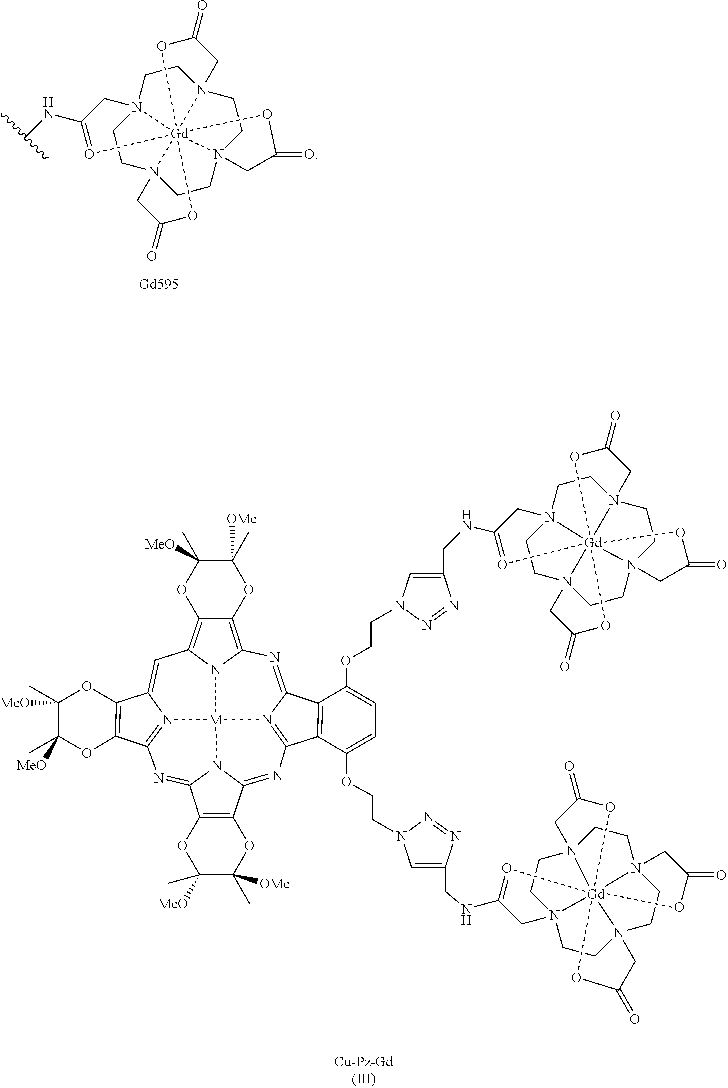

The present non-invasive pz-Gd conjugates for use as diagnostic probes for tumor detection provide an important improvement in patient diagnosis and post-therapy monitoring of a cancer reemergence and, accordingly, a reduction in mortality from cancer. Tumor diagnosis typically is performed clinically using X-ray procedures that expose patients to harmful ionizing radiation. MRI is a safe procedure, but there is difficulty distinguishing between healthy and diseased tissues and the extent of tumor margins without the use of a contrast agent. The present compounds are contrast agents that localize selectively within tumors and act as an MRI contrast agent for initial tumor diagnosis or treatment monitoring. The present pz-Gd conjugates also exhibit NIR absorption/emission, and are preferentially accumulated and retained in tumor cell lines in vitro, as opposed to native cell lines.

The present porphyrazine-Gd(III) conjugates act as an MRI contrast agents for the detection of cancer. The present components exhibit cellular uptake in breast tumor cells, as demonstrated by an MR image of these cells in vitro. Mice with xenograft breast tumors were administered a present compound intravenously and tumor regions appeared brighter by MRI after injection.

In particular, an amphiphilic Cu(II) porphyrazine (pz)-Gd(III) DOTA conjugate of the present invention was prepared and used as an MRI contrast agent. Experiments show that the present pz-Gd(III) conjugates exhibit intense optical absorbance and fluorescence in the NIR window with optimum tissue penetration; b) are selectively taken up by tumor cells and (c) localize in tumors in vivo. The Cu-Pz-Gd(III) conjugate was synthesized in good yield and exhibited solution phase molecular relaxivity (r₁=23.0) sufficient for MRI. Breast tumor cells (MDA-MB-231) took up Cu-Pz-Gd(III) in vitro where significant contrast enhancement is observed in phantom cell pellet MR images. The compound was administered in vivo to an orthotopic breast tumor model in athymic nude mice. The average T₁ of the tumor regions in mice (n=3) treated with 50 mg/kg Cu-Pz-Gd(III) decreased relative to saline treated controls, and the agent was rapidly cleared, primarily through the kidneys. Disparate T₁ enhancement by Cu-Pz-Gd(III) in the tumor's core relative to the periphery offers evidence that the conjugate agent has application in the imaging of necrotic tissue.

### Relaxivity and Octanol-Water Partition Coefficient

The efficiency of a contrast agent in reducing the longitudinal relaxation time of water protons is defined in terms as relaxivity (mM⁻¹ s⁻¹) (14). It has been predicted by Solomon-Bloembergen-Morgan theory that a slow molecule tumbling of an MRI agent causes an increase in rotational correlation time, Tᵣ, which in turn results in increase in relaxivity (14, 15). A common approach to increase relaxivity by slowing down the molecular tumbling is to attach Gd(III) complexes to a macromolecule through rigid linkages (12,16). Longitudinal relaxivity (r₁) measurements on Zn-Pz-Gd(III) and Cu-Pz-Gd(III) are summarized in Table 1. The relaxivity of the monomeric DOTA-Gd(III) (DOTA is 1,4,7,10-tetraazacyclododecane-N,N,N″,N″-tetraacetic acid) used in the clinic is shown for comparison (17). Zn-Pz-Gd(III) showed an ionic relaxivity of 6.9 mM⁻¹ s⁻¹ per Gd(III), whereas Cu-Pz-Gd(III) exhibited an enhanced ionic relaxivity of 11.5 mM⁻¹ s⁻¹. Both the Zn-Pz-Gd(III) and Cu-Pz-Gd(III) had higher longitudinal relaxivity than DOTAGd(III).

TABLE 1
Relaxivity and partition coefficient of
M—Pz—Gd(III) complexes.

| | DOTA—Gd | Zn—Pz—Gd | Cu—Pz—Gd |
|---|---|---|---|
| | (III) | (III) | (III) |
| MW (g/mol) | 561.4 | 2230.5 | 2229.5 |
| Ionic r₁ (mM⁻¹ S⁻¹) | 3.1 | 6.9 | 11.5 |
| No. of Gd(III) | 1 | 2 | 2 |
| Molecular r₁ | 3.1 | 13.8 | 23.0 |
| (mM⁻¹ S⁻¹) | | | |
| log P | −3.42 | −3.13 | −2.41 |

Octanol-water partition coefficients (log P) were measured by ICP-MS to determine the hydrophilicity of the Pz-Gd(III) conjugates (4d, 18). The hydrophilicity of a molecule dictates cellular permeability where a balance in amphiphilicity is desired for ease of aqueous administration and maximum cellular uptake (19). Cu-Pz-Gd(III) is slightly more hydrophobic than Zn-Pz-Gd(III) and both conjugates are more hydrophobic than DOTA-Gd(III).

### In Vitro Cellular Uptake and MRI of M-Pz-Gd(III) Contrast Agents

Breast tumor cells (MDA-MB-231) were treated with M-Pz-Gd(III) complexes at varying concentrations for 24 hours, washed, digested in acid, and Gd(III) content was measured by ICP-M S (FIG. 1). An M-Pz-Gd(III) dose of 50 μM corresponds to an overall Gd(III) dose of 100 μM because two Gd(III) ions are present per molecule. Cells also were treated with an equivalent amount of DOTA-Gd(III), an agent known not to cross cell membranes. Cellular uptake of Zn-Pz-Gd(III) is similar to that of DOTA-Gd(III), indicating that this complex does not enter cells effectively. However, Cu-Pz-Gd(III) exhibits a two-fold increase in Gd(III) content per cell indicating the compound crosses cell membranes.

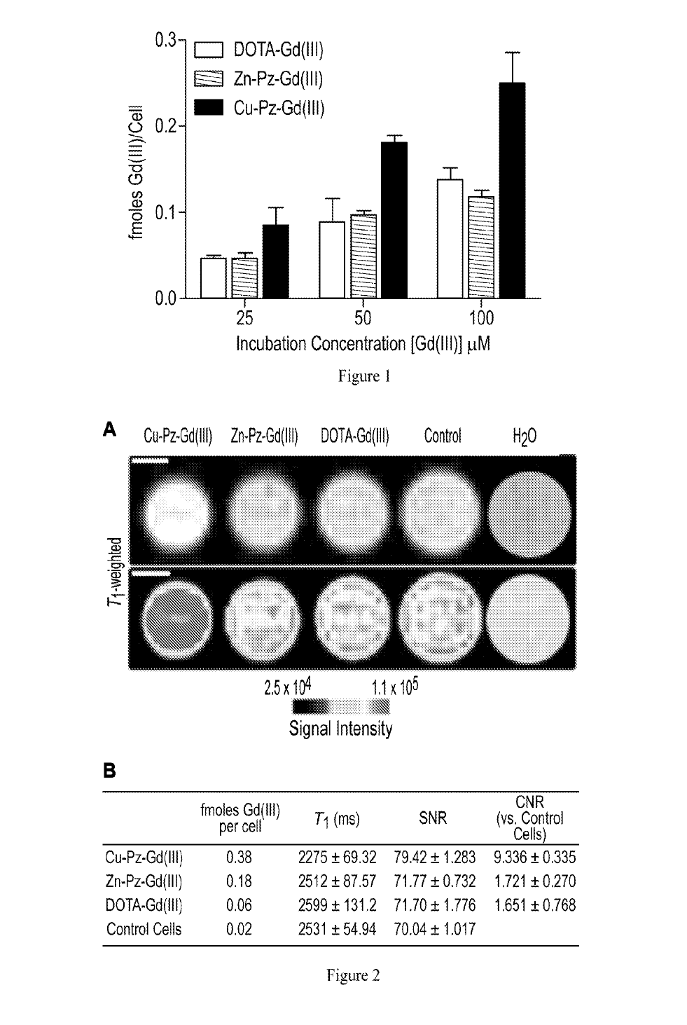

To determine whether the cellular uptake and molecular relativity of Cu-Pz-Gd(III) provides an effective MR contrast agent, MDA-MB-231 breast tumor cells were treated with M-Pz-Gd(III) complexes (200 μM) and centrifuged into capillaries (about 1.0 mm diameter) prior to MR imaging. The grayscale and color intensity MR images of the cell pellets show significant increases in image intensity (which represents a decrease in T₁) for cells treated with Cu-Pz-Gd(III) compared with cells treated with either Zn-Pz-Gd(III), DOTA-Gd(III), or vehicle controls (FIG. 2A). The contrast to noise ratio (CNR) was found to be 9.336. The amount of Gd(III) per cell was determined by ICP-MS for each pellet and, as expected, T₁ is inversely proportional to Gd(III) content. (FIG. 2B).

### Time Course In Vivo Tumor MRI in Mice and Biodistribution

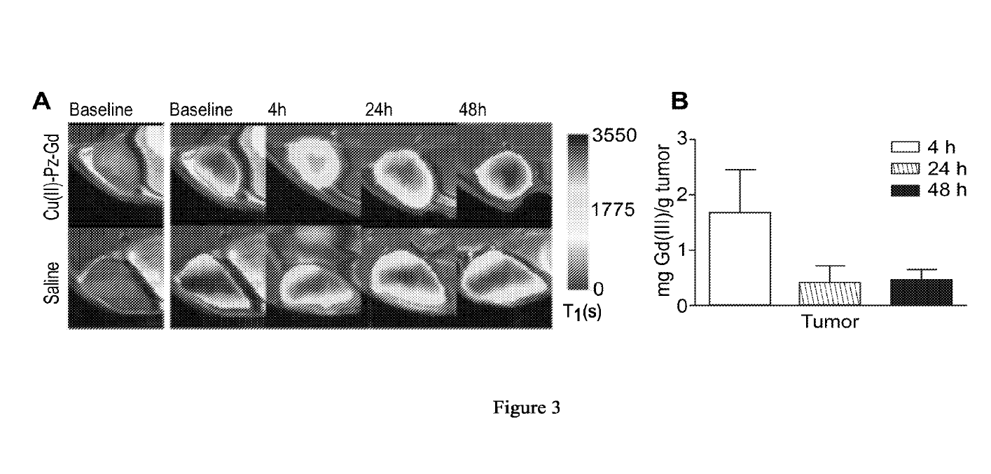

Cu-Pz-Gd(III) was examined as a contrast agent in vivo in an orthotopic breast tumor model in athymic nude mice. MDA-MB-231 breast tumor cells expressing mCherry fluorescent protein were implanted in the mammary fat pad of mice and tumors were allowed to grow to about 5 mm in diameter at which time mice were treated with 450 nmol (50 mg/kg) Cu-Pz-Gd(III) together with saline treated control mice (FIG. 3A). MR images of tumors in treated mice exhibited significant contrast enhancement at the 4 hour timepoint. After 24 hours, Cu-Pz-Gd(III) had been cleared considerably from the tumors (FIG. 3B) and, based on biodistribution data measured by ICP-M S, was being cleared from the mice primarily through the kidneys and liver. Urine collected from mice was green in color and HPLC analysis indicates that the conjugate was cleared intact.

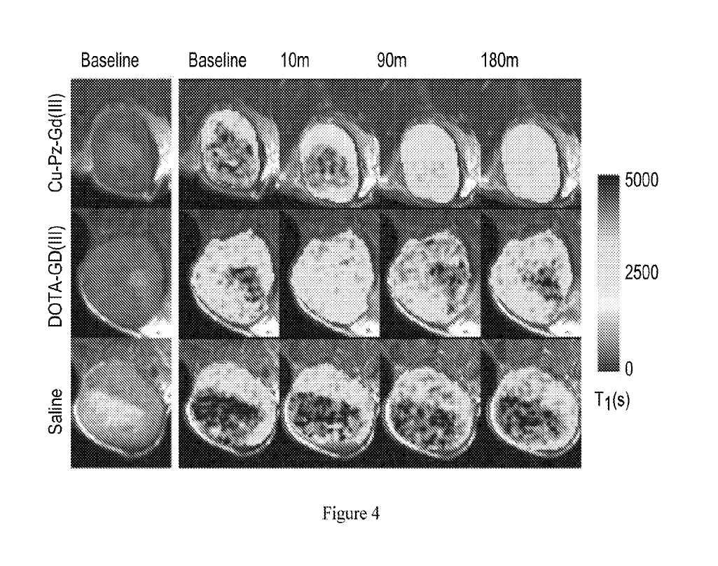

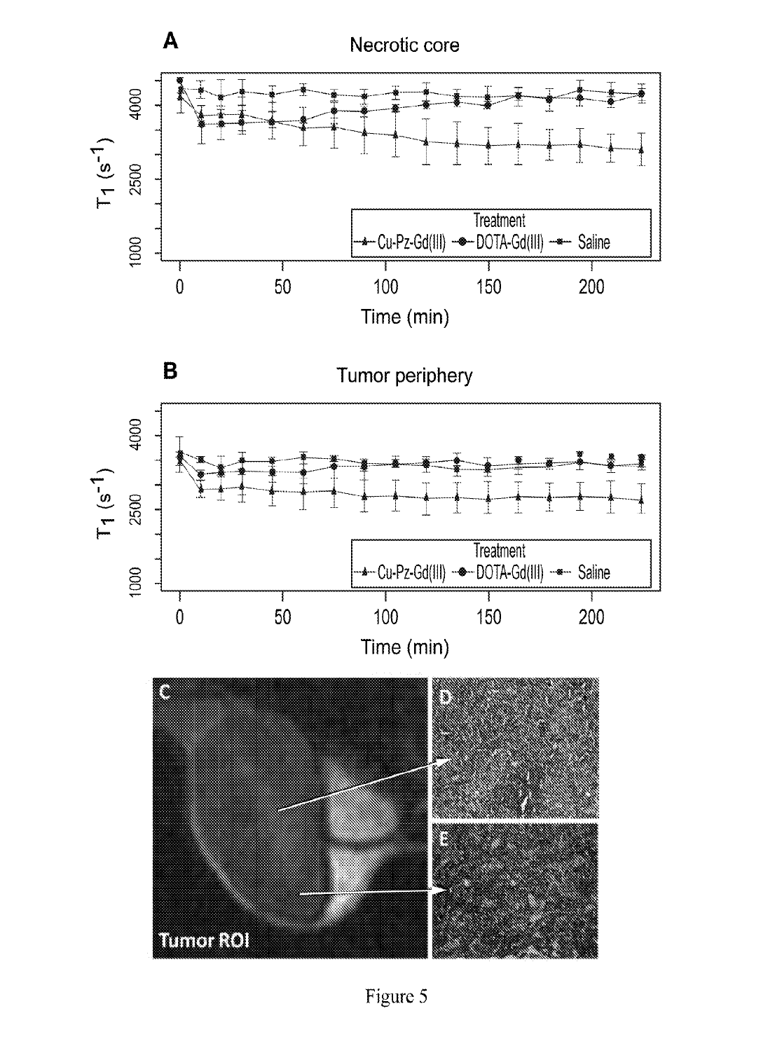

In a separate experiment focusing only on short time points (0-225 min), groups of mice (n=3) were treated with Cu-Pz-Gd(III) (450 nmol, 50 mg/kg), DOTA-Gd(I I I) (900 nmol, 25 mg/kg), and saline as a negative control (FIG. 4). Mice were imaged continuously after tail vein injections. Whereas DOTA-Gd(III) shows an initial decrease in tumor T₁ followed by recovery of signal, Cu-Pz-Gd(III) is slower to achieve the same effect, but the decrease in T₁ is much more persistent. Regions of interest (ROIs) were defined outside the periphery of tumors, at the inner rim, and at the center. Plots of T₁ over time for these regions demonstrate that these are three distinct regions. The non-tumor periphery region had a much lower T₁, which did not change over the course of the experiment. The outer rim of the tumor exhibited significant T₁ shortening that levels off while the center of the tumor underwent slower T₁ shortening (FIG. 5A vs. 5B), eventually being roughly equivalent to the rim of the tumor (t=135 min) This effect was not observed for DOTA-Gd(III) treated mice, where the T₁ of ROI s went essentially unchanged.

To determine whether these differences in ROI represented real differences in the tumor tissue, the tumor of an untreated mouse was collected and cryosectioned for histological analysis (FIG. 5C-5E). At the center of the tumor, many fenestrations existed that did not appear at the tumor periphery. This difference in morphology most likely represents the onset of necrosis at the tumor core and could explain the differential in relative T₁ of ROIs discussed above.

Pz 285 was used as a platform for preparing and testing second-generation Gd(III) MRI contrast agents because: a) functional groups for conjugation to pz 285 lie on one side of the pz periphery, preventing appended hydrophilic Gd(III) complexes from interfering with hydrophobic interactions between the pz and transport proteins; and b) pz 285 is produced in extremely high synthetic yields for a tetrapyrrolic macrocycle (45%) on a multi-gram scale, making biological studies and large-scale manufacture economical. As previously demonstrated, utilizing robust alkyne-azide cycloaddition (“click”) reactions for conjugation bypassed the disadvantages of other synthetic schemes. However, metallation of the pz with Zn or Cu, which required for this conjugation chemistry because it uses a copper catalyst, effectively quenches pz fluorescence due to the heavy atom effect and d-electron spin-orbit coupling. The slight paramagnetism of Cu(II) that prevents Cu-Pz-Gd(III) from being a multimodal fluorescent/MR contrast agent enhances the complex's MR effect by increasing relaxivity (r₁) relative to Zn-Pz-Gd(III). This effect was observed in analogous pz-Gd(III) conjugates but was much less pronounced (12).

The paramagnetism of Cu(II) adds to the relaxivity of the Cu agent, but the differential hydrophobicity is likely more important. The Cu of a Cu-pz lies in the plane of the tetrapyrrole ring and does not coordinate an axial ligand, whereas the Zn(II) ion sits 0.31 Å out of the basal plane (20) and binds with a water molecule, making the pz macrocycle more hydrophilic. This can result in the partial aggregation of the Cu-Pz-Gd(III) conjugate, which leads to slower molecular tumbling rate and higher longitudinal relaxivity.

Breast tumor cells were used for in vitro uptake studies because MDA-MB-231 cells produce excellent orthotopic tumor models in mice. The major finding from these in vitro studies, that much more Cu-Pz-Gd(III) conjugate is taken up by cells than Zn-Pz-Gd(III) conjugate or DOTA-Gd(III), is in agreement with an unrelied upon hypothesis that the hydrophobic metallo-macrocycle of the Cu-pz conjugate is responsible for cellular uptake. In accordance with this hypothesis, the increased cellular uptake of Cu-Pz-Gd(III) correlates well with the octanol-water partition coefficient measurements.

Cell pellet MR imaging confirmed that the combination of enhanced uptake of Cu-Pz-Gd(III) and its high molecular relaxivity warranted investigation in vivo. In vivo evaluation of Cu-Pz-Gd(III) reveals that the contrast agent accumulates in tumor tissue at short time points and is cleared through the kidneys and liver. However, Cu-Pz-Gd(III) uptake persists after the hydrophilic monomeric DOTA-Gd(III) is cleared. This is consistent with the hypothesis that Cu-Pz-Gd(III) is actively being taken up by cells rather than simply acting as a vascular agent. The presence of distinct regions within the tumor (i.e., rim vs. center) indicates that Cu-Pz-Gd(III) may localize preferentially within necrosis at the tumor core and these tumor regions show distinct histological differences consistent with the onset of necrosis. There is literature directed to this phenomenon among similar porphyrin-Gd(III) conjugates (8b, 9d), illustrating the use of Cu-Pz-Gd(III) as a necrosis imaging agent useful for cardiovascular imaging (21).

Zn-Pz-Gd(III) and Cu-Pz-Gd(III) conjugates have been prepared via cycloaddition reactions between an azide functionalized pz and alkyne functionalized DOTA-Gd(III) analog. Relaxivity and hydrophobicity of these complexes are higher than the monomeric clinical Gd(III) contrast agent with Cu-Pz-Gd(III) exhibiting the most promising physical properties. This leads to excellent cellular uptake and MR contrast enhancement for Cu-Pz-Gd(III), both in vitro and in vivo. A disparate contrast enhancement between the center and rim of the tumors was observed, indicating that the success of the Cu-Pz-Gd(III) conjugate in decreasing T₁ of tumor regions may be due to localization within necrosis.

### Materials and Methods

### General Methods

Unless otherwise noted, materials and solvents were purchased from Sigma-Aldrich Chemical Co. (St. Louis, Mo.) and used without further purification. Gd595 (MW 595 g/mol) was prepared according to literature (16a, 22). Unless noted, all reactions were performed under a nitrogen or argon atmosphere. Acetonitrile was purified using a Glass Contour Solvent system (Pure Process Technology, Nashua, N.H., USA). Deionized water (18.2 MΩ·cm) was obtained from a Millipore Q-Guard System (Billerica, Mass.). Thin-layer chromatography (TLC) was performed on EMD 60F 254 silica gel plates. Standard grade 60 Å 230-400 mesh silica gel (Sorbent Technologies, Norcross, Ga., USA) was used for flash column chromatography. ¹H and ¹³C NMR spectra were obtained on a Bruker 500 MHz Avance III NMR spectrometer (Bruker Biospin, Billerica, Mass., USA) or a Varian Inova 400 MHz NMR spectrometer (Agilent Technologies, Santa Clara, Calif., USA) with deuterated solvents as noted. Elixtrospray ionization mass spectrometry (ESI-MS) was carried out using a Varian 1200L single-quadrupole mass spectrometer (Agilent Technologies, Santa Clara, Calif., USA). Matrix-assisted laser desportion ionization time-of-flight mass spectra (MALDI-TOF-MS) were recorded on a Bruker AutFlex III (Bruker, Billerica, Mass., USA), using 2,5-dihydroxybenzoic acid as the matrix. Analytical reverse-phase HPLC-MS was performed on a Varian Prostar 500 system (Agilent Technologies, Santa Clara, Calif., USA) using a Waters (Milford, Mass., USA) Atlantis C18 column (4.6×250, 5 μm). This system is equipped with a Varian 380 LC ELSD system, a Varian 363 fluorescence detector, a Varian 335 UVvis detector, and a Varian 1200L quadrupole MS detector (Agilent Technologies, Santa Clara, Calif., USA). Preparative runs were performed on a Waters (Milford, Mass., USA) Atlantis C18 column (19×250, 10 um). The mobile phase consisted of water (solvent A) and HPLC-grade acetonitrile (solvent B).

### 18,21-bis(2-azidoethoxy)-tris-[1,4-dioxinok,l,q1(2R,3R)-2,3-dimethoxy-2,3-dimethyll 25H,27H-benzo[b] porphyrazine 288

Pz 286 (100 mg, 0.08 mmol, 1 eq) was dissolved in DMF (20 mL) and NaN₃ (104 mg, 1.6 mmol, 20 eq) in water (10 mL) was added at room temperature. The mixture was heated at 90° C. for 72 hours. Rotary evaporation and extraction separation between water and dichloromethane gave the crude product as blue solid. Purification of chromatography was performed on silica gel (eluent 40:1 CH₂Cl₂: MeOH) yielding the pure product (61 mg, 79%). ¹H NMR (500 MHz, CDCl3) δ 2.07 (s, 611), 2.13 (s, 611), 2.14 (s, 611), 3.51 (s, 611), 3.54 (s, 6H), 3.62 (s, 6H), 4.29 (m, 2H), 4.49 (m, 2H), 5.06 (t, 4H), 7.66 (s, 2H); ¹³C NM R (125 MHz, CDCl3) δ 157.3, 151.2, 150.8, 138.7, 136.8, 135.3, 135.0, 130.7, 120.9, 102.4, 102.0, 71.2, 51.2, 50.1, 50.0, 49.9, 29.7, 18.0, 17.9; ESI-MS calcd. For C₄₂H₅₂N₁₄O₁₄: [M]⁺ 976.38. found: 976.58.

### Zn-Pz-Gd(III) Zn[Pz(C₁₇H₂₇N₅O₇Gd)₂]. Pz=18,21-bis(2-(1H-1,2,3-triazole-4-yl) ethoxy)-tris-[1,4-dioxino[g,l,q](2R,3R)-2,3-dimethoxy-2,3-dimethyll 25H,27H-benzo[b]porphyrazine; C₁₇H₂₇N₅O₇Gd=N-methyl-2-aminoacetamide-4,7,10-tris(carboxymethyl)-1,4,7,10-tetraazacyclododecylgadolinium(III))

Pz azide 288 (20 mg, 0.009 mmol) and 10 equiv of zinc chloride were dissolved in 5 ml DMF. The solution was stirred at room temperature under nitrogen for 48 hours. The mixture was evaporated to dryness under reduced pressure, dissolved in CH₂Cl₂, and washed twice with H₂O. The organic phase was concentrated and dried under vacuum overnight. The intermediate was dissolved in 20 mL of water/DMF (1:1) with Gd595 (16 mg, 0.027 mmol), copper sulfate (3 mg, 0.018 mmol), and sodium ascorbate (7 mg, 0.036 mmol). The reaction mixture was heated in an oil bath at 65° C. for 48 hours. After cooling, the mixture was evaporated to dryness and purified by reverse phase HPLC according to method 1, retention time of 39.5 minutes, to afford a dark green powder (10.5 mg, 52.3%). MALDI-TOF-MS m/z 2228.4 (M+H⁺) calcd for C₈₀H₁₀₆Gd₂N₂₄O₂₈Zn 2230.3.

### Cu-Pz-Gd(III) Cu[Pz(C₁₉H₂₈N₈O₇Gd)₂]. Pz=18,21-bis(2-(1H-1,2,3-triazole-4-yl) ethoxy)-tris-[1,4-dioxino[g,l,q](2R,3R)-2,3-dimethoxy-2,3-dimethyll 25H,27H-benzo[b]porphyrazine; C₁₉H₂₈N₈O₇Gd=N-methyl-2-aminoacetamide-4,7,10-tris(carboxymethyl)-1,4,7,10-tetraazacyclododecylgadolinium(III))

Pz azide 288 (20 mg, 0.009 mmol) was dissolved in 20 mL of water/DMF (1:1) with Gd595 (16 mg, 0.027 mmol), copper sulfate (16 mg, 0.10 mmol), and sodium ascorbate (35 mg, 0.18 mmol). The reaction mixture was heated in an oil bath at 65° C. for 48 hours. After cooling, the mixture was evaporated to dryness and purified by reverse phase HPLC according to method 1, retention time of 43.4 minutes, to afford a dark green powder (11.5 mg, 57.3%). MALDI-TOF-MS m/z 2227.3 (M+H⁺) calcd for C₈₀H₁₀₆Gd₂N₂₄O₂₈Cu 2229.5.

### Relaxation Time Measurements

Solutions of M-Pz-Gd(III) (M=Zn, Cu) conjugates were dissolved in 500 μL of filtered, deionized H₂0 (18.2 MΩ·cm) for T₁ acquisition. Relaxation times were measured on a Bruker mq60 NMRanalyzer equipped with Minispec v2.51 Rev.00/NT software (Bruker Biospin, Billerica, Mass., USA) operating at 1.41 T (60 MHz) and 37° C. T₁ relaxation times were measured using an inversion recovery pulse sequence (T₁_{_{—}}ir_mb) using the following parameters: 4 scans per point, 10 data points for fitting, mono-exponential curve fitting, phase cycling, 10 ms first pulse separation, and a recycle delay and final pulse separation≧5 T₁. The gadolinium concentrations of each solution were determined using ICP-MS. The inverse of the longitudinal relaxation time (1/ T₁, s⁻¹) was plotted against gadolinium concentration (mM) and fitted to a straight line with an R²>0.99. The slope of the fitted line was recorded as the relaxivity, r₁.

### Octanol-Water Partition Coefficients

DOTA-Gd(I I I), Zn-Pz-Gd(III), and Cu-Pz-Gd(III) (0.5 mg) were dissolved in 1 mL of a 1:1 mixture of water:1-octanol, respectively. After shaking for 30 seconds, these tubes were placed on a rotator for gentle mixing to equilibrate for 48 hours. Gd(III) concentration in each layer was determined by ICP-MS. Partition coefficients were calculated from the equation log P=log(Cₒ/C_{w}), where log P is the logarithm of the partition coefficient, Cₒ is the concentration of Gd in the 1-octanol layer, and C_{w} is the concentration of Gd in the water layer.

### Cell Counting Using a Guava EasyCyte Mini Personal Cell Analyzer (PCA) System

After cell harvesting, an aliquot of the cell suspensions was mixed with Guava ViaCount reagent and allowed to stain at room temperature for at least 5.0 minutes (no longer than 20 minutes). Stained samples were vortexed for 10 seconds and counted. Percent cell viability was determined via manual analysis using a Guava EasyCyte Mini Personal Cell Analyzer (PCA) and ViaCount software module. For each sample, 1000 events were acquired with dilution factors that were determined based upon optimum machine performance (about 25-70 cells/μL). Instrument reproducibility was assessed daily using GuavaCheck Beads and following the manufacturer's suggested protocol using the Daily Check software module.

### MR Imaging and T₁/T₂ Analysis of Cell Pellets at 9.4 T

9.4 T MR imaging and T₁/T₂ measurements were performed on a Bruker Biospec 9.4 T imaging spectrometer fitted with shielded gradient coils at 25° C. For cell pellets images, about 2.0×10⁶ MDA-MB-231 cells were incubated in 25-cm² T-flasks with either Cu-Pz-Gd(III), Zn-Pz-Gd(III) or DOTA-Gd(III) (400 μM wrt Gd(III)) for 24 hours, rinsed with DPBS (3×5 mL/flask), and harvested with 500 μL, of a 0.25-vol % trypLE Express solution. After addition of 500 μL of the appropriate fresh complete media (1.0 mL total volume/flask), cells were transferred to 1.5-mL microcentrifuge tubes and centrifuged at 200 g for 5 minutes. The supernatant was removed; the cell pellets then were re-suspended in 1 mL of complete media, added to 5¾″ flame-sealed Pasteur pipets, and centrifuged at 200 g and 4.0° C. for 5 minutes. The bottom sections of the flame-sealed pipets were then scored with a glass scribe, broken into small capillaries, then imaged using a RF RES 400 1H 089/038 quadrature transmit receive 23-mm volume coil (Bruker BioSpin, Billerica, Mass., USA).

Spin-lattice relaxation times (T₁) were measured using a rapid-acquisition rapid-echo (RARE-VTR) T₁-map pulse sequence, with static TE (10 ms) and variable TR (100, 200, 400, 500, 750, 1000, 2500, 5000, 7500, and 10000 ms) values. Imaging parameters were as follows: field of view (FOV)=25×25 mm², matrix size (MTX)=256×256, number of axial slices=3, slice thickness (SI)=1.0 mm, and averages (NEX)=4 (total scan time=2 hrs 58 min) T₁ analysis was carried out using the image sequence analysis tool in Paravision 5.0 p13 software (Bruker, Billerica, Mass., USA) with mono-exponential curve-fitting of image intensities of selected regions of interest (ROIs) for each axial slice.

Spin-spin relaxation times (T₂) were measured using a multi-slice multi-echo (MSME) T₂-map pulse sequence, with static TR (6000 ms) and 64 fitted echoes in 10 ms intervals (10, 20 and 640 ms). Imaging parameters were as follows: field of view (FOV)=25×25 mm², matrix size (MTX)=256×256, number of axial slices=3, slice thickness (SI)=1.0 mm, and averages (NEX)=4 (Total scan time=1 hr 16 min) T₂ analysis was carried out using the image sequence analysis tool in Paravision 5.0 p13 software (Bruker, Billerica, Mass., USA) with mono-exponential curve-fitting of image intensities of selected regions of interest (ROIs) for each axial slice.

### Animal Model

MDA-MB-231 cells (1×10⁶) expressing mCherry fluorescent protein were inoculated subcutaneously (1:1 v/v matrigel:PBS) on the right mammary fat pad. Cells were maintained in DMEM media supplemented with 10% FBS prior to inoculation. The athymic nude mice, Crl:NU(NCr)-Foxnl^{nm}, were purchased from Charles River, Portage at 5-6 weeks old and 16-18 gram were allowed to acclimate for 5 days. One week post inoculation, each animal was screened for mCherry fluorescence wavelength using the IVIS small animal fluorescence imaging system to verify presence of the tumor.

### In Vivo MR Imaging at 9.4 T

Mice were anesthetized in an induction chamber with 3% inhaled isoflurane in oxygen, then transferred to an imaging bed with circulating warm water heating system, MRI compatible respiratory monitoring (SA Instruments, Stony Brook, N.Y.) and 1-2% isoflurane delivered via nosecone to maintain a surgical plane of anesthesia with respiratory rate at 90-100 breaths/min Mice were imaged prone with the tumor centered in a 40 mm diameter quadrature volume coil (Bruker Biospec, Billerica, Mass.). An AutoPac automated positioning system was used to reproducibly center the animals in the MRI system.

### 48 Hour Timecourse Study

Mice were positioned in the MRI scanner as described above and localizer images were acquired. T₁ maps were acquired using a FAIR-RARE non-selective inversion recovery sequence with static TR (18,000 ms) and TE (6.1 ms), and variable TI (30.5, 100, 200, 300, 500, 600, 700, 800, 900, 1000, 1100, 1200, 1300, 1400, 1600, 2000, 2500, 3000, 3500, 4000, 4500, 5000, and 6000 ms). Imaging parameters were as follows: field of view (FOV)=40×20 mm², matrix size(MTX)=512×133 pixels, number of slices=1, slice thickness=1.0 mm, and 1 average (total scan time=5 m 26 s).

Images were acquired at baseline, and 4, 24, and 48 hours after injection of 150 μL of imaging agent via the tail vein. One control animal was injected with saline; the remaining 9 animals were injected with 450 nmol Cu-Pz-Gd(III). Three animals were sacrificed after 4 hour, 24 hour, and 48 hour timepoints, respectively; the organs were collected for ICP-MS (described below) to measure biodistribution.

### 4 Hour Timecourse Study

Mice were positioned in the MRI scanner as described above and localizer images were acquired. To improve signal to noise ratio, reduce respiratory artifacts, and increase tumor coverage, for this study we used a multi slice T₁ map acquired using a variable repetition time saturation recovery sequence (RARE-VTR) with static TE (8.2 ms) and variable TR (175, 300, 600, 800, 1200, 2000, 4000 ms). Imaging parameters were as follows: FOV=10×7.5 mm, MTX=80×60, number of axial slices=5, slice thickness=1.0 mm, and 1 average (total scan time=6 min 48 sec). A T₂ map was acquired using a multi echo spin echo sequence (MSME) with TR=3000 and TE=10-320 ms in increments of 10 ms. The imaging table was then ejected from the scanner and 150 uL of imaging agent was injected intravenously via the tail vein without moving the animal. The imaging agents included Cu-Pz-Gd(III) (450 nmol), DOTA-Gd(III) (900 nmol), or saline. After injection, the imaging table was returned to the center of the scanner using the AutoPac system. A rapid localizer image was used to confirm accurate repositioning, followed by T₁ maps acquired 10, 20, and 30 minutes post injection, and every fifteen minutes thereafter for a total of 3.5 hours. T₂ maps were acquired 37 minutes after injection and every thirty minutes thereafter. Mice were removed from the MRI scanner after 3.5 hours and sacrificed immediately, with tissue collection as described below.

### MRI Data Analysis

Data analysis was performed using the JIM software (Xinapse Systems, Aldwincle, UK) in conjunction with Java utilities written in-house using the Xinapse API. Upon examination of the images, it became apparent that there were two distinct tumor zones with different patterns of agent uptake, namely a liquid-filled central region with long T₂, and a peripheral rim of solid tissue. Regions of interest (ROIs) were drawn on these two zones based on T₂ maps. T₁ maps were constructed based on the central three slices to avoid the partial-volume effects apparent on slices 1 and 5 in most tumors. To avoid fitting noise, the image background was masked out using an automated particle analysis algorithm ROIs were copied from the T₂ weighted images to the T₁ maps for timecourse analysis, with slight manual adjustment as needed to compensate for a small amount of motion over the course of the 3.5 hour imaging session. At each timepoint, T₁ was averaged for each ROI over the three center image slices, weighted by ROI area. Average T₁ values over the experimental groups, along with standard deviations, were calculated and plotted against time for each of the two tumor zones using R (23).

### Tissue Collection

Mice were sacrificed by CO₂ inhalation followed by cervical dislocation. Blood, urine (where possible), tumor, liver, kidney, spleen, heart, lungs, and samples of skin and skeletal muscle were collected for elemental analysis by ICP-MS as described below. In one additional mouse that was not treated with an imaging agent, the tumor was collected and fixed in 10% formalin for histological analysis.

### Quantification of Gadolinium Via Inductively Coupled Plasma Mass Spectrometry

Quantification of Gd was accomplished using ICP-MS of acid digested samples. Solution samples were digested in ACS reagent grade nitric acid (70%, Sigma, St. Louis, Mo., USA) and incubated in a water bath at 70° C. for at least 2 hours to allow for complete sample digestion. A portion of the digested sample was added to a 15 mL conical tube along with 5 ng/mL of multi-element internal standard containing Bi, Ho, In, Li, Se, Tb, and Y (Inorganic Ventures, Christiansburg, Va., USA) and filtered, de-ionized H₂O (18.2 MΩ·cm). Instrument calibration was accomplished by preparing individual-element Gd standard (Inorganic Ventures, Christiansburg, Va., USA) using concentrations of 1.000, 5.000, 10.00, 20.00, 50.00, 100.0, and 200.0 ng/mL containing 3.0% nitric acid (v/v) and 5.0 ng/mL of multi-element internal standard.

For organ digestion (biodistribution) teflon tubes were boiled in a mixture of about 1-5% Alconox (w/v) and 3.0% (v/v) ACS reagent grade nitric acid (70%) to ensure complete removal of lipid and residual gadolinium. The tubes were then washed with filtered, de-ionized H₂O (18.2 MΩ·cm) and dried in an oven for at least 4 hours at 80° C. Organs were weighed and put into clean Teflon tubes followed by the addition of 1 mL of ACS reagent grade nitric acid (70%) per one gram of tissue. Samples were digested in a Milestone EthosEZ microwave digestion system (Shelton, Conn., USA) with a 120° C. temperature ramp for 20 minutes, 120° C. hold for 20 minutes, followed by a 40 minute cool down cycle. The resultant liquefied organ samples were then weighed with a portion of each sample being placed in a clean pre-weighed 15 mL conical tube followed by addition of multi-element internal standard and filtered, de-ionized H₂0 (18.2 MΩ·cm) to produce a final solution of 3.0% nitric acid (w/w) and 5 ng/mL internal standard up to a total sample volume of 5 mL.

ICP-M S was performed on a computer-controlled (Plasmalab software) Thermo X series II ICP-MS (Thermo Fisher Scientific, Waltham, Mass., USA) operating in standard mode equipped with an ESI 50-2 autosampler (Omaha, Nebr., USA). Each sample was acquired using 1 survey run (10 sweeps) and 3 main (peak jumping) runs (100 sweeps). The isotopes selected for analysis were ^{(157, 158)}Gd with ⁽¹¹⁵⁾In and ⁽¹⁶⁵⁾Ho isotopes selected as internal standards for data interpolation. Instrument performance is optimized daily through an autotune followed by verification via a performance report (passing manufacturer specifications). Addition of all reagents for all samples and standards were weighed using a Mettler Toledo (Columbus, Ohio, USA) X5205 DeltaRange analytical micro balance (with 0.01 mg resolution for up to 81 g of sample).

The pz-Gd(III) conjugates of the present invention find use in imaging of tumors, including, but not limited to, breast, lung, skin, testicular, and other upper aerodigestive tumors, and as simultaneous anti-tumor agents through photodynamic therapeutic applications. The pz-Gd(III) conjugates of the present invention find use as MR and NIR imaging agents. The pz-Gd(III) conjugates of the present invention find further use as therapeutic agents and simultaneous imaging/therapeutic agents whose therapeutic effects occur with light activation (photodynamic therapy).

In one embodiment, the present invention provides methods of treating cancerous tumors comprising the administration of a present pz-Gd(III) conjugate with light activation in conjunction with recognized anti-tumor modalities of surgery, radiotherapy, and chemotherapy. The effectiveness of a treatment can be measured in clinical studies or in model systems, such as a tumor model in mice or cell culture sensitivity assays. The present invention provides a combination therapy that results in improved effectiveness and/or reduced toxicity. Accordingly, in one embodiment, the invention relates to the use of a present pz-Gd(III) conjugate in conjunction with, surgery, radiotherapy or chemotherapy. In some embodiments, particularly when M is 2H or Mg, a present pz-Gd(III) conjugate also can be used with light activation in a method of treating a cancerous tumor.

When the combination therapy of the invention comprises administering a present pz-Gd(III) conjugate with one or more additional anticancer agents, the pz-Gd(III) conjugate and the additional anticancer agents can be administered to an individual concurrently or sequentially. The agents can also be cyclically administered. Cycling therapy involves the administration of one or more anticancer agents for a period of time, followed by the administration of one or more different anticancer agents for a period of time and repeating this sequential administration, i.e., the cycle, in order to reduce the development of resistance to one or more of the anticancer agents of being administered, to avoid or reduce the side effects of one or more of the anticancer agents being administered, and/or to improve the efficacy of the treatment.

An additional anticancer agent may be administered over a series of sessions; any one or a combination of the additional anticancer agents listed below may be administered.

The present invention includes methods for treating cancer comprising administering to an individual in need thereof a present pz-Gd(III) conjugate and one or more additional anticancer agents or pharmaceutically acceptable salts thereof. The pz-Gd(III) conjugate and the additional anticancer agent can act additively or synergistically. Suitable anticancer agents include, but are not limited to, gemcitabine, capecitabine, methotrexate, taxol, taxotere, mereaptopurine, thioguanine, hydroxyurea, cyclophosphamide, ifosfamide, nitrosoureas, mitomycin, dacarbazine, procarbizine, etoposide, teniposide, campatheeins, bleomycin, doxorubicin, idarubicin, daunorubicin, dactinomycin, plicamycin, mitoxantrone, L-asparaginase, doxorubicin, epirubicin, 5-fluorouracil (5-FU), taxanes (such as docetaxel and paclitaxel), leucovorin, levamisole, irinotecan, estramustine, etoposide, nitrogen mustards, BCNU, nitrosoureas (such as carmustine and lomustine), platinum complexes (such as cisplatin, carboplatin and oxaliplatin), imatinib mesylate, hexamethylmelamine, topotecan, tyrosine kinase inhibitors, tyrphostins herbimycin A, genistein, erbstatin, and lavendustin A.

In one embodiment, the anti-cancer agent can be, but is not limited to, a drug selected from the group consisting of alkylating agents, nitrogen mustards, cyclophosphamide, trofosfamide, chlorambucil, nitrosoureas, carmustine (BCNU), lomustine (CCNU), alkylsulphonates, busulfan, treosulfan, triazenes, plant alkaloids, vinca alkaloids (vineristine, vinblastine, vindesine, vinorelbine), taxoids, DNA topoisomcrase inhibitors, epipodophyllins, 9-aminocamptothecin, camptothecin, crisnatol, mitomycins, mitomycin C, anti-metabolites, anti-folates, DHFR inhibitors, trimetrexate, IMP dehydrogenase inhibitors, mycophenolic acid, tiazofurin, ribavirin, EICAR, ribonuclotide reductase inhibitors, hydroxyurea, deferoxamine, pyrimidine analogs, uracil analogs, floxuridine, doxifluridine, ratitrexed, cytosine analogs, cytarabine (ara C), cytosine arabinoside, fludarabine, purine analogs, mercaptopurine, thioguanine, DNA antimetabolites, 3-HP, 2′-deoxy-5-fluorouridine, 5-HP, alpha-TGDR, aphidicolin glycinate, ara-C, 5-aza-2′-deoxycytidine, beta-TGDR, cyclocytidine, guanazole (inosine glycodialdehyde), macebecin II, pyrazoloimidazole, hormonal therapies, receptor antagonists, anti-estrogen, tamoxifen, raloxifene, megestrol, LHRH agonists, goserelin, leuprolide acetate, anti-androgens, flutamide, bicalutamide, retinoids/deltoids, cis-retinoic acid, vitamin A derivative, all-trans retinoic acid (ATRA-IV), vitamin D3 analogs, E1) 1089, CB 1093, ICH 1060, photodynamic therapies, vertoporfin, BPD-MA, phthalocyanine, photosensitizer Pc4, demethoxy-hypocrellin A (2BA-2-DMHA), cytokines, interferon-a, interferon-I3, interferon-y, tumor necrosis factor, angiogenesis inhibitors, angiostatin (plasminogen fragment), antiangiogenic antithrombin UI, angiozyme, ABT-627, Bay 12-9566, benefin, bevacizumab, BMS-275291, cartilage-derived inhibitor (CDI), CAI, CD59 complement fragment, CEP-7055, Col 3, combretastatin A-4, endostatin (collagen XVIII fragment), fibronectin fragment, Gro-beta, halofuginone, heparinases, heparin hexasaccharide fragment, HMV833, human chorionic gonadotropin (hCG), IM-862, interferon inducible protein (IP-10), interleukin-12, kringle 5 (plasminogen fragment), marimastat, metalloproteinase inhibitors (UMPs), 2-methoxyestradiol, MMI 270 (CGS 27023A), MoAb IMC-I C11, neovastat, NM-3, panzem, P1-88, placental ribonuclease inhibitor, plasminogen activator inhibitor, platelet factor-4 (PF4), prinomastat, prolactin 161(D fragment, proliferin-related protein (PRP), PTK 787/ZK 222594, retinoids, solimastat, squalamine, SS 3304, SU 5416, SU 6668, SU 11248, tetrahydrocortisol-S, tetrathiomolybdate, thalidomide, thrombospondin-1 (TSP-1), TNP-470, transforming growth factor-beta (TGF-11), vasculostatin, vasostatin (calreticulin fragment), ZD 6126, ZD 6474, famesyl transferase inhibitors (FTI), bisphosphonates, antimitotic agents, allocolchicine, halichondrin B, colchicine, colchicine derivative, dolstatin 10, maytansine, rhizoxin, thiocolchicine, trityl cysteine, isoprenylation inhibitors, dopaminergic neurotoxins, 1-methyl-4-phenylpyridinium ion, cell cycle inhibitors, staurosporine, actinomycins, actinomycin D, dactinomycin, bleomycins, bleomycin A2, bleomycin B2, peplomycin, anthracycline, adriamycin, epirubicin, pirarnbicin, zorubicin, mitoxantrone, MDR inhibitors, verapamil, Ca²′ATPase inhibitors, and thapsigargin.

Other anti-cancer agents that may be used in the present invention include, but are not limited to, acivicin; aclarubicin; acodazole hydrochloride; acronine; adozelesin; aldesleukin; altretamine; arnbomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelcsin; bleomycin sulfate; brequinar sodium; bropirimine; busul fan; cactinomycin; calusterone; caracemide; carbetimer; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexorrnaplatin; dezaguanine; dezaguanine mesylate; diaziquone; docetaxel; doxorubicin hydrochloride; droloxifene; droloxifene citrate; dromostanolone propionate; duazomycin; edatrexate; eflomithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-Ia; interferon gamma-Ib; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mecchlorethamine hydrochloride; megestrol acetate; melengestrol acetate; melphalan; menogaril; mercaptopurine; methotrexate sodium; metoprine; meturedepa; mitindomide; mitocarcin; mitocromin; mitogillin; mitomalcin; mitomycin; mitusper; mitotane; mitoxantrone hydrochloride; mycophenolic acid; nocodazole; nogalamycin; ormaplatin; oxisuran; pegaspargase; peliomycin; pentamustine; peplomycin sulfate; perfosfarnide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsornycin; spirogermanium hydrochloride; spiromustine; spiroplatin; streptonigrin; streptozocin; sulofenur; talisomycin; tecogalan sodium; tegafur; teloxantrone hydrochloride; temoporfin; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracit mustard; uredepa; vapreotide; verteporfln; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozolc; zeniplatin; zinostatin; zorubicin hydrochloride.

Further chemotherapeutic agents that can be used in the present invention include, but are not limited to: 20-epi-1,25-dihydroxyvitamin D3; 5-ethynyluracil; abiraterone; aclarubicin; acylfulvene; adecypenol; adozelesin; aldesleukin; ALL TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein 1; antiandrogen, prostatic carcinoma; antiestrogen; antineoplaston; antisense oligonucleotides; aphidicolin glycinate; apoptosis gene modulators; apoptosis regulators; apurinic acid; ara CDP DL PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCRJABL antagonists; benzochlorins; benzoylstaurosporine; beta lactam derivatives; beta alethine; betaclarnycin B; betulinic acid; bFGF inhibitor; bicalutamide; bisantrene; bisaziridinylsperrnine; bisnafide; bistratene A; bizelesin; breflate; bropirimine; budotitane; buthionine sulfoximine; calcipotriol; calphostin C; camptothecin derivatives; canarypox IL-2; carboxamide amino triazole; carboxyarnidotriazole; CaRest M3; CARN 700; cartilage derived inhibitor; carzelesin; casein kinase inhibitors; castanospermine; cecropin B; cetrorelix; chlorins; chloroquinoxaline sulfonamide; cicaprost; cis porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexveraparnil; diaziquone; didemnin B; didox; diethylnorspermine; dihydro 5 azacytidine; dihydrotaxol, 9; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflomithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fltidarabine; fluorodaunoruniein hydrochloride; forfenimex; formestane; fostriecin; fotemustine; gadolinium texaphyrin; gallium nitrate; galocitabine; ganirelix; gelatinase inhibitors; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin like growth factor 1 receptor inhibitor; interferon agonists; interferons; interleukins; iobenguane; iododoxorubiein; ipomeanol, 4; iroplact; irsogladine; isobengazole; isohomohalicondrin B; itasetron; jasplakinolide; kahalalide F; larnellarin N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum complexes; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1 based therapy; mustard anti-cancer agent; mycaperoxide B; mycobacterial cell wall extract; myriaporone; N acetyldinaline; N substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06 benzylguanine; octreotide; okicenone; oligonucleotides; onapristone; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum complexes; platinum triamine complex; porfimer sodium; porfiromycin; prednisone; propyl his acridone; prostaglandin J2; proteasome inhibitors; protein A based immune modulator; protein kinase C inhibitor; protein kinase C inhibitors, microalgal; protein tyrosine phosphatase inhibitors; purine nucleoside phosphorylase inhibitors; purpurins; pyrazoloaeridine; pyridoxylated hemoglobin polyoxyethylene conjugate; raf antagonists; raltitrexed; ramosetron; ras farnesyl protein transferase inhibitors; ras inhibitors; ras GAP inhibitor; retelliptine demethylated; rhenium Re 186 etidronate; rhizoxin; ribozymes; RH retinamide; rogletimide; rohitukine; romurtide; roquinimex; rubiginone BI; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer.

In the present methods, a therapeutically effective amount of a present pz-Gd(III) conjugate, typically formulated in accordance with pharmaceutical practice, is administered to a human being in need thereof. A present pz-Gd(III) conjugate can be administered by any suitable route.

Pharmaceutical compositions include those wherein a present pz-Gd(III) conjugate is present in a sufficient amount to be administered in an effective amount to achieve its intended purpose. The exact formulation, route of administration, and dosage is determined by an individual physician in view of the specific tumor of interest.

The pz-Gd(III) conjugates of the present invention typically are administered in admixture with a pharmaceutical carrier selected with regard to the intended route of administration and standard pharmaceutical practice. Pharmaceutical compositions for use in accordance with the present invention are formulated in a conventional manner using one or more physiologically acceptable carriers comprising excipients and auxiliaries that facilitate processing and administration of the pz-Gd(III) conjugate.

The term “carrier” refers to a diluent, adjuvant, or excipient, with which a present pz-Gd(III) conjugate is administered. Such pharmaceutical carriers can be liquids, such as water and oils, including those of petroleum, animal, vegetable or synthetic origin, such as peanut oil, soybean oil, mineral oil, sesame oil, and the like. In addition, auxiliary, stabilizing, thickening, and lubricating agents can be used. The pharmaceutically acceptable carriers are sterile. Water is a preferred carrier when the pz-Gd(III) conjugate is administered intravenously. Saline solutions and aqueous dextrose and glycerol solutions can also be employed as liquid carriers, particularly for injectable solutions. Suitable pharmaceutical carriers also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The present compositions, if desired, can also contain minor amounts of wetting or emulsifying agents, or pH buffering agents.

These pharmaceutical compositions can be manufactured, for example, by conventional mixing, dissolving, emulsifying, entrapping, or lyophilizing processes. Proper formulation is dependent upon the route of administration chosen. When administered in liquid form, a liquid carrier, such as water, can be added. The liquid form of the composition can further contain physiological saline solution, dextrose or other saccharide solutions, or glycols. When administered in liquid form, the composition contains about 0.1% to about 90%, and preferably about 1% to about 50%, by weight, of a present pz-Gd(III) conjugate.

When a therapeutically effective amount of a present pz-Gd(III) conjugate is administered by intravenous, cutaneous, or subcutaneous injection, the composition is in the form of a pyrogen-free, parenterally acceptable aqueous solution. The preparation of such parenterally acceptable solutions, having due regard to pH, isotonicity, stability, and the like, is within the skill in the art. A preferred composition for intravenous, cutaneous, or subcutaneous injection typically contains, an isotonic vehicle. The pz-Gd(III) conjugate can be infused with other fluids over a 10-30 minute span or over several hours.

A present pz-Gd(III) conjugate can be formulated for parenteral administration by injection, e.g., by bolus injection or continuous infusion. Formulations for injection can be presented in unit dosage form, e.g., in ampules or in multidose containers, with an added preservative. The compositions can take such forms as suspensions, solutions, or emulsions in oily or aqueous vehicles, and can contain formulatory agents such as suspending, stabilizing, and/or dispersing agents.

Pharmaceutical compositions for parenteral administration include aqueous solutions of the active agent in water-soluble form. Additionally, suspensions of a present pz-Gd(III) conjugate can be prepared as appropriate oily injection suspensions. Suitable lipophilic solvents or vehicles include fatty oils or synthetic fatty acid esters. Aqueous injection suspensions can contain substances which increase the viscosity of the suspension. Optionally, the suspension also can contain suitable stabilizers or agents that increase the solubility of the compounds and allow for the preparation of highly concentrated solutions. Alternatively, a present composition can be in powder form for constitution with a suitable vehicle, e.g., sterile pyrogen-free water, before use.

As an additional embodiment, the present invention includes kits which comprise one or more compounds or compositions packaged in a manner that facilitates their use to practice methods of the invention. In one simple embodiment, the kit includes a compound or composition described herein as useful for practice of a method (e.g., a composition comprising a pz-Gd(III) conjugate and an optional second therapeutic agent), packaged in a container, such as a sealed bottle or vessel, with a label affixed to the container or included in the kit that describes use of the compound or composition to practice the method of the invention. Preferably, the compound or composition is packaged in a unit dosage form. The kit further can include a device suitable for administering the composition according to the intended route of administration, for example, a syringe, drip bag, or patch. In another embodiment, pz-Gd(III) conjugate is a lyophilate. In this instance, the kit can further comprise an additional container which contains a solution useful for the reconstruction of the lyophilate.

### REFERENCES

1. (a) X. P. Hu et al., Annual Review of Biomedical Engineering 2004, 6, 157-184; (b) L. Frullano et al., Journal of Biological Inorganic Chemistry 2007, 12 (7), 939-949.
2. (a) S. J. Lord et al., European Journal of Cancer 2007, 43 (13), 1905-1917; (b) E. A. Morris, Seminars in Roentgenology 2001, 36 (3), 226-237; (c) D. J. Brenner et al., American Journal of Roentgenology 2001, 176 (2), 289-296; (d) J. F. Paul et al., European Radiology 2007, 17 (8), 2028-2037.
3. (a) C. D. Lehman et al., New England Journal of Medicine 2007, 356 (13), 1295-1303; (b) C. Kuhl, Radiology 2007, 244 (2), 356-378; (c) M. Morrow et al., Lancet 2011, 378 (9805), 1804-1811; (d) M. Kriege et al., New England Journal of Medicine 2004, 351 (5), 427-437.
4. (a) M. V. Knopp et al., Molecular Cancer Therapeutics 2003, 2 (4), 419-426; (b) D. E. Sosnovik et al., Current Opinion in Biotechnology 2007, 18 (1), 4-10; (c) J. Lee et al., Journal of The American Chemical Society 2005, 127 (38), 13164-13166; (d) P. A. Sukerkar, et al., Molecular Pharmaceutics 2011, 8 (4), 1390-1400; (e) S. Iwaki et al., Bioorganic & Medicinal Chemistry Letters 2012, 22 (8), 2798-2802.
5. (a) C. Colosimo et al., European Radiology 1997, 7, S231-S237; (b) W. Krause, International Journal of Neuroradiology 1998, 4 (4), 296-312; (c) T. P. L. Roberts et al., European Journal of Radiology 2000, 34 (3), 166-178.
6. (a) H. Marom et al., Journal of Medicinal Chemistry 2010, 53 (17), 6316-6325; (b) M. Q. Tan et. al, Pharmaceutical Research 2012, 29 (4), 953-960; (c) S. R. Bull et al., Nano Letters 2005, 5 (1), 1-4.
7. N. Kamaly et al., Organic & Biomolecular Chemistry 2010, 8 (1), 201-211.
8. (a) M. Ethirajan et al., Chemical Society Reviews 2011, 40 (1), 340-362; (b) Y. Ni, Current Medical Imaging Reviews 2008, 4 (2), 96-112.
9. (a) S. W. Young et al., Proceedings of The National Academy of Sciences of The United States of America 1996, 93 (13), 6610-6615; (b) C. P. Gros et al., Medchemcomm 2011, 2 (2), 119-125; (c) G. L. Li et al., Bioconjugate Chemistry 2005, 16 (1), 32-42; (d) S. Metz et al., Academic Radiology 2003, 10 (5), 484-490.
10. (a) M. J. Fuchter et al., Australian Journal of Chemistry 2008, 61 (4), 235-255; (b) S. Lee et al., Metal-based drugs 2008, 3914-3918; (c) E. R. Trivedi et al., Photochemistry and Photobiology 2010, 86 (2), 410-417.
11. E. R. Trivedi et al., Proceedings of The National Academy of Sciences of The U.S. Pat. No. 2,010,107 (4), 1284-1288.
12. Y. Song et al., Bioconjugate Chemistry 2010, 21 (12), 2267-2275.
13. E. R. Trivedi et al., Tetrahedron Letters 2012, 53 (41), 5475-5478.
14. A. E. Merbach et al., The Chemistry of Contrast Agents in Medical Magnetic Resonance Imaging. Wiley: New York, 2001.
15. E. Toth et al., Contrast Agents I 2002, 221, 61-101.
16. (a) D. E. Prasuhn et al., Chemical Communications 2007, (12), 1269-1271; (b) A. Datta et al., Journal of The American Chemical Society 2008, 130 (8), 2546-2552; (c) Y. Song et al., Journal of The American Chemical Society 2008, 130 (21), 6662-+.
17. Y. Song et al., Angewandte Chemie-International Edition 2009, 48 (48), 9143-9147.
18. F. A. A. Nunez et al., Journal of Pharmaceutical Sciences 1997, 86 (10), 1187-1189.
19. (a) A. Leo et al., Chemical Reviews 1971, 71 (6), 525-+; (b) P. D. Leeson et al., Nature Reviews Drug Discovery 2007, 6 (11), 881-890.
20. (a) A. G. Montalban et al., Journal of Organic Chemistry 1997, 62 (26), 9284-9289; (b) M. Zhao et al. Journal of The American Chemical Society 2005, 127 (27), 9769-9775.
21. J. Barkhausen et al., Journal of The American College of Cardiology 2002, 39 (8), 1392-1398.
22. R. F. H. Viguier et al., Journal of The American Chemical Society 2006, 128 (35), 11370-11371.
23. R Development Core Team R. A language and environment for statistical computing, R Foundation for Statistical Computing: Vienna, Austria, 2010.

1. A porphyrazine-gadolinium (III) conjugate having a structure: 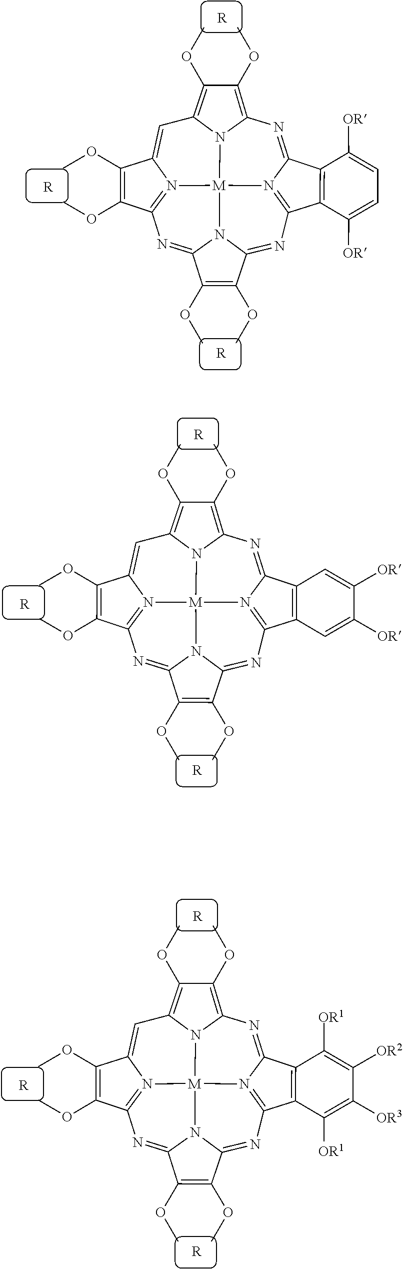
wherein M is 2H, Cu, Mg, or Zn; 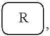
independently, is 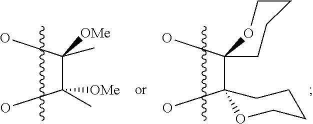
R′ is 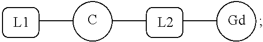
R¹ and R², each independently, are R′, OC₁₋₈alkyl, C₃₋₈cycloalkyl, O(CH₂CH₂O)ₙOH, or O(CH₂CH₂O)ₙO(C₁₋₄alkyl); 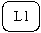
is (CH₂)ₙ, (CH₂CH₂O)ₙ, OOC(CHOH)ₙCOO, COO(CH₂)ₙOOC, or CON(CH₂)ₙNOC, wherein n is 1 through 10; 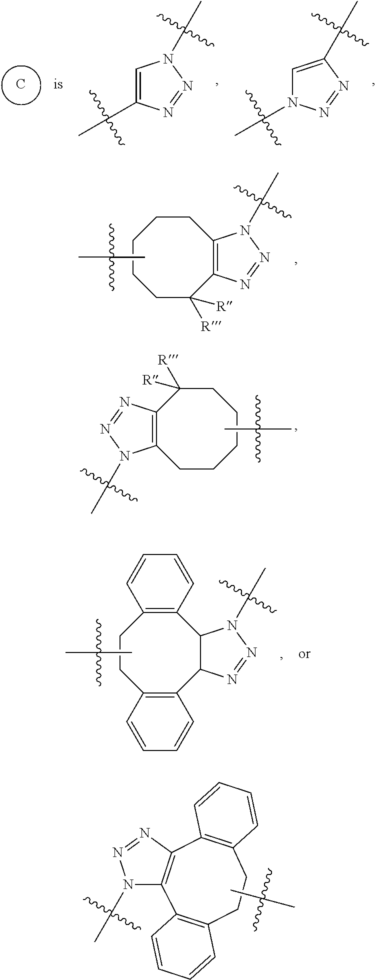
wherein R″ and R′″, independently, are H or F; and

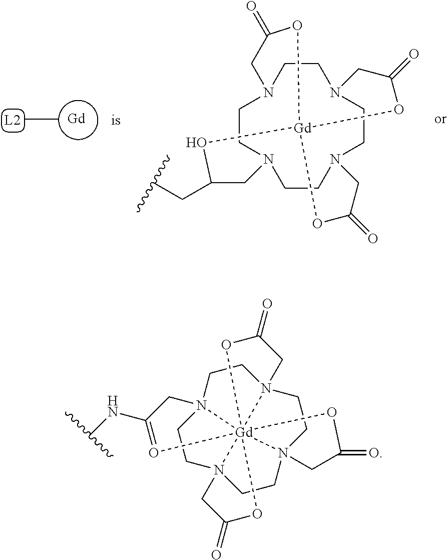

2. A porphyrazine-gadolinium (III) conjugate of claim 1 having a structure:

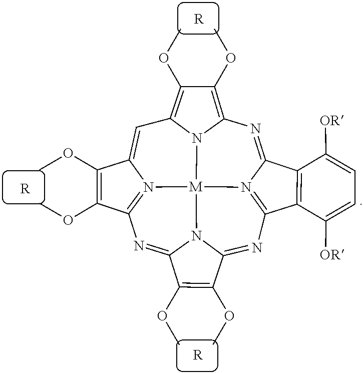

3. The conjugate of claim 1 wherein R is

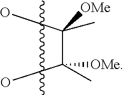

4. The compound of claim 1 wherein L1, independently, is (CH₂)ₙ or (CH₂CH₂O)ₙ, and n is 1-5.

5. The compound of claim 1 wherein C, independently, is

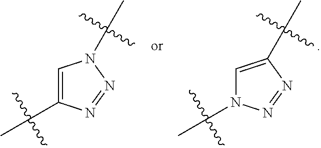 [SMILES: *c1cn(*)nn1 ; *c1cn(*)nn1]

6. The compound of claim 1 wherein L2-Gd is

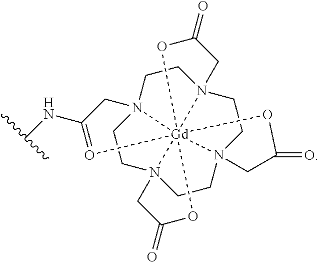

7. A compound having a structure

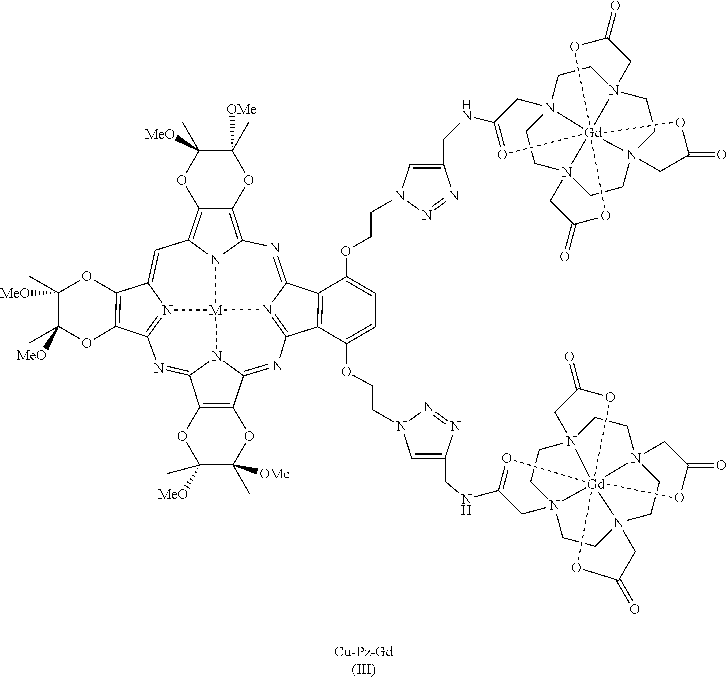

8. A method of imaging a tumor comprising:
A. administering a sufficient amount of a porphyrazine-gadolinium (III) of claim 1 for visualization to a mammal suspected of having a tumor; and
B. visualizing the porphyrazine in the mammal,
wherein the porphyrazine localizes at a tumor in the mammal.

9. The method of claim 8 wherein the visualizing is selected from the group consisting of NIR imaging and MR imaging.

10. The method of claim 9 wherein NIR imaging and MR imaging are jointly or simultaneously used after administration of the porphyrazine-gadolinium (III) conjugate.

11. The method of claim 8 wherein the mammal is a human.

12. A method of killing tumor cells comprising administering a therapeutically effective amount of a porphyrazine-gadolinium (III) conjugate of claim 1 to an individual in need thereof, and light activating the porphyrazine-gadolinium (III) conjugate.

13. The method of claim 12 further comprising administration of a therapeutically effective amount of a chemotherapeutic agent, radiation, or both.
